(19) United States
(12) Patent Application Publication
(10) Pub. No.: US 2003/0129080 A1
(43) Pub. Date: Jul. 10, 2003

(54) Stable, non-hazardous indicia for biocidal irradiation of a package

(75) Inventors: David F. Lewis, Monroe, CT (US); J. Michael Donahue, Morris, NY (US); Carl A. Listl, New Hyde Park, NY (US); Xiang Yu, Bridgewater, NJ (US)

Correspondence Address: Attn: William J. Davis, Esq., INTERNATIONAL SPECIALTY PRODUCTS, Legal Department, Building No. 10, 1361 Alps Road, Wayne, NJ, 07470, US

(73) Assignee: ISP INVESTMENTS INC.

(21) Appl. No.: 10/295,523

(22) Filed: Nov. 15, 2002

Related U.S. Application Data
(60) Provisional application No. 60/333,298, filed on Nov. 19, 2001.

(51) Int. Cl. G05B 1/00; A61L 2/00

(52) U.S. Cl. 422/22; 422/3; 422/119; 422/186; 250/455.11

(57) ABSTRACT

An item of mail or other container having an irradiation target area containing a radiochromic polyacetylene monomer indicia which is substantially stable to incident light, humidity and ambient temperatures and which develops a darkened color phase alteration at a level of radiation exposure sufficient to deactivate a bio-hazardous agent and a method of applying said radiochromic polyacetylene to a package or container.

### CROSS REFERENCE TO RELATED APPLICATIONS

This application corresponds to U.S. Provisional Application Serial No. 60/333,298, filed on Nov. 19, 2001, the entire disclosure of which is incorporated herein by reference.

### FIELD OF THE INVENTION

This invention relates to packages imprinted with color alterable indicia which is irreversible with respect to radiation exposure generated by gamma rays, X-rays, electrons, neutrons, ultraviolet photons or other exposure generated at a wavelength less than 700 nm for the purpose of determining the efficacy of irradiation treatment against harmful pathogens such as bacteria, viruses and the like.

### BACKGROUND OF THE INVENTION

It is known that items of mail and other packages or containers can be irradiated to inactivate hazardous biological agents. As a means for determining that irradiation or a proper dosage of irradiation has occurred, it is desirable that the item be visibly marked so that handlers can be assured of its safety.

To avoid false positive indication of antimicrobial irradiation, the indicia material should be inactive with respect to normal environmental conditions of heat, humidity and light to which the item may be exposed during collection, sorting, transportation and delivery. Conversely, the positive indication, visually observed by a distinct color alteration, should be irreversible with respect to irradiation needed to inactivate harmful agents and should remain visually apparent over extended periods associated with normal handling, delivery and storage. Finally, the indicia material should be environmentally safe and innocuous to human contact or exposure.

In accordance with the above criteria, it is an object of this invention to provide a marking on the exterior surface of the package, such as indicia in the form of a visually distinctive stamp, label, transfer, ink or crayon marking or dye, which is imprinted or affixed at the time the article dispatched.

Another object is to provide indicia comprising a component that is stable under normal conditions of incident light, humidity and heat but which undergoes, with respect to irradiation, an irreversible color change upon exposure to a level of energy sufficient to inactivate harmful microscopic agents.

Another object is to regulate and identify the effective dosage by means of indicia on certain packages so as to avoid harmful affects on their contents, for example, pharmaceuticals, films, videos and the like, or to detoxify pathogens which may be contained within a package such as a pathogen which may be contained in packaged blood, blood components, food and the like, or to alter the genetic or biological function of a species, e.g. an insect species.

Still another object is to provide a means for determining the dosage of irradiation on a package by intercalation between color intensity and dosage.

Yet another object is to provide an item of mail or other container clearly printed with a message to indicate whether protective irradiation has been effected.

It is also an object of the invention to disclose an economical method for preparing the present indicia and for applying it to a package, for example an item of mail or container subject to human contact.

These and other advantages and benefits of the invention will become apparent from the following description and disclosure.

### SUMMARY OF THE INVENTION

In accordance with this invention there is provided an indicia of irradiation in a predefined target area of the package which essentially comprises the use of a crystalline, radiochromic, conjugated polyacetylene that is capable of undergoing a radiation-irreversible darkened color change upon exposure at a dosage level of from about 0.001 to about 100,000 grey (Gy), preferably 1 to 50,000 Gy, from a source of radiation with an energy between about 1 KeV and about 100 MeV, preferably 1-10 MeV. The polyacetylene monomer in a molten state or, in a liquid mixture is applied directly onto the package in a specified target area or indirectly by means of an irradiatable label or stamp affixed to the package. The polyacetylene monomer can be dispersed or dissolved in an inert liquid carrier, such as water, an alcohol, glycol, a wax or mixture of waxes or any mixture of the foregoing carriers to provide a suitable printing ink or spray. Desirably, the liquid composition containing the polyacetylene has a viscosity between about 0.1 and about 10,000 cp, preferably between about 0.5 and about 50 cp, and can be applied by means of an inkjet printer where the dosage of polyacetylene can be monitored to the requirements of a particular package or group of packages or by the use of an ink stick or a spray gun optionally in combination with a stencil, to provide a desired marking or message.

### DETAILED DESCRIPTION OF THE INVENTION

The crystalline, conjugated, radiochromic, image developing polyacetylene monomer component of the present invention contains from 12 to 60 carbon atoms and is described by the formula:

A-(CH₂)ₘ—(C≡C—)ₚ—(CH₂)ₙ—B

wherein m and n each independently have a value of from 0 to 30; p has a value of 2 to 4; A and B each independently are R, OR₁, OH, COOR₂, CONR₃R₄ or (CH₂)ᵣ—O—CO—NR₅R₆ or a metal salt of the acid or ester; and where R, R₁, R₂, R₃, R₄, R₅ and R₆ are each independently hydrogen or C, to C₁₂ alkyl or aryl and r has a value of from 1 to 4. Also mixtures of the above monomers can be employed.

Representative examples of specific polyacetylene monomers include pentacosa-10,12-diynoic acid, tricosa-10,12-diynoic acid, heneicosa-10,12-diynoic acid and metal salts thereof; eicosa-5,7-diyn-1-ol, 13,15-octacosadiyne; 5,7-dodecodiyne-1,12-bis(butyl carbamate); 5,7-dodecodiyne-1,12-bis(n-propyl carbamate); 5,7-dodecodiyne-1,12-bis-(isopropyl carbamate). Of these, pentacosa-10,12-diynoic acid and 5,7-dodecodiyne-1,2-bis(n-butyl carbamate) exhibit high sensitivity to the radiation exposure requirements of this invention.

The color alteration of the polyacetylene of this invention is associated with intermolecular addition polymerization between adjacent —C≡C—C≡C— functionalities. The effective phase change can be visually determined by an immediate color darkening thereby providing a progressive gradation of darkened color or shades of color, depending upon the intensity and duration of irradiation exposure. For example, within a 10 second exposure to a 10 KeV X-ray source, at a dosage of 100 Gy/sec., a darkening of the indicia is equal to exposure for 1000 seconds from a 10 KeV X-ray source at a dosage rate of 1 Gy/sec.

Although the colorless, polyacetylene monomer may be employed in a molten state, for field use, the active monomer, or mixture of monomers, is preferably dispersed in crystalline form, preferably microcrystalline form, in the inert carrier.

To prepare the polyacetylene composition, the selected polyacetylene component is uniformly mixed with the liquid carrier and combined in a weight ratio of between about 0.1:99.9 and about 99:1, preferably between about 1:99 and about 90:10, to form a solution or microdispersion readily adapted for printing.

Specific examples of suitable liquid carriers for the polyacetylene or polyacetylene mixtures include water, ethylene- or propylene-glycol, aliphatic and aromatic alcohols, cyclohexanol, N-methylpyrrolidone, or natural or synthetic waxes such as beeswax, carnauba, candelwax, bayberry, candelilla, paraffin, polyesters, polyolefins, polyamides and the like. Mixtures of the above carriers can also be employed.

Before combining with the carrier, crystalline polyacetylene monomer can be dissolved in a suitable solvent, preferably a volatile solvent such as acetone, ethanol, propanol, ethyl acetate, etc. for the purpose of recrystallization and promotion of a microcrystalline dispersion. However, the solvent is removed by evaporation, absorption or other means before imaging in order to restore the polyacetylene monomer to its effective crystalline state.

The polyacetylene monomer composition may also contain a dye. The function of the dye is either to provide better contrast in the color differentiation or to enhance the light stability of the polyacetylene product. For example, yellow dye can be blended with the colorless polyacetylene monomer. Upon irradiation, the foresaid blend changes to a blue or black color due to polymerization of the polyacetylene monomer. Magenta, red or other colored and/or UV absorbing dyes can be used separately or in combination, or added in mixtures with the yellow dye to further enhance the polyacetylene monomer stability. Accordingly, certain dyes or dye mixtures may be selected for their ability to absorb UV or light in wavelengths to which a certain polyacetylene monomer may be sensitive. In this manner, the polyacetylene monomer is protected against incidental and environmental exposure to those wavelengths.

The polyacetylene composition optionally may contain up to 90 wt. %, preferably 0.1 to 50 wt. % of other inactive excipients such as an activator, a metal ion chelating agent, an opacifying agent, an anti-oxidizing agent, a surfactant, a thickening agent, a binder or a mixture of these. The purpose of an activator is to increase the absorption of radiation of a particular energy or wavelength and to transfer the energy to the radiation sensitive polyacetylene. Exemplary of such activators are metal compounds, particularly metal halides, containing one or more elements with an atomic number >18. Suitable metal halides include Group I halides such as cesium chloride, bromide or iodide and rubidium chloride, bromide or iodide and the like.

Representative metal ion chelating or sequestering agents are disodium ethylenediaminetetraacetate; sodium oxalate; citric acid; sodiumtartrate; sodium polyphosphate; potassium hypophosphate; sodium diethyldithiocarbamate and sodium salts of N,N,N′,N′-ethylenediaminetetra-(methylenephosphonic acid) and 1-hydroxyethane-1,1-diphosphonic acid and combinations thereof.

Examples of opacifying agents include insoluble metal oxides, carbonates, sulfates, sulfites, sulfides, carboxylates, phosphates and silicates. Propyl gallate, sodium diethyldithiocarbonate, citric acid, sodium citrate, ascorbic acid, alkali metal sulfides and silfites, 3-tert-butyl-4-hydroxy-5-methylphenyl sulfide, butylated hydroxytoluene, butylated hydroxyanisole, tert-butylhydroquinone, hydroxylamine and hydroxylamine hydrochloride are examples of suitable anti-oxidants which may be included in the composition at a concentration of up to 5 wt. % with respect to the polyacetylene monomer. Any of the conventional gums may also be included in the composition for thickening purposes. Useful binders include water-soluble natural and synthetic polymers, gelatin, carboxymethyl cellulose, polyacrylic acid, polyvinyl alcohols, polyvinyl pyrrolidone and thermal plastic materials such as natural and synthetic waxes.

The polyacetylene can be applied to a substrate by the use of a printer, e.g. a wax transfer printer or other device. In such applications, the polyacetylene monomer can be heated above its melting point or it can be dissolved in a polyacetylene monomer solubilizing agent. The resulting solution can then be then mixed with the liquid carrier and any desired excipients added and mixed until a uniform composition is formed. The solvent, when one is used, is removed before imaging so as to restore the crystalline state of the polyacetylene monomer.

The printer transfers the radiation sensitive polyacetylene monomer composition directly to a target area on the package or to a separate stamp or label which is affixed to the package. In one particular embodiment, a message is imprinted on an item of mail, package, envelope or carton, or on a label affixed thereto. For example the word “IRRADIATED” is clearly printed in an area which is insensitive to irradiation and in an adjacent, polyacetylene sensitive area, printed with the word “NOT”; so that upon irradiation, the word “NOT” becomes obliterated by the darkened color of the irradiated polymerized polyacetylene monomer. It will be understood that other messages can be similarly imprinted on an article subject to irradiation.

In another aspect of the invention, the dosage of irradiation can be determined by the depth of darkening achieved by irradiation when shades of color development are keyed to particular dosage amounts. For example, at radiation within the lower limits of the above range, a noticeable and somewhat darker color is obtained, so that, by grades of color intensity, it is possible to quantitatively gauge the degree of treatment or dosage commensurate with that required for inactivation of a particular pathogen or to limit the dosage of certain packages containing sensitive contents. In the latter case, exposure with a lower irradiation dosage may be employed.

Having generally described the invention, reference is now had to the following examples which illustrate various best modes of application but which are not to be construed as limiting to the scope of the invention as defined in the appended claims.

### EXAMPLE 1

In a glass container, 13 grams of pentacosa-10,12-diynoic acid (PCDA) was introduced and mixed with 13 grams of a printer ink block consisting of a mixture of waxes and a transparent yellow dye. The resulting mixture was heated in an oven at 120° C. for a period of 10 minutes and then filtered to remove any polymerized PCDA and the filtrate, having a viscosity of less than 10 cps was poured into a mold adapted to feed the yellow ink slot of a Tektronix Phaser 300 printer operated at 125° C. The printer was used to imprint a yellow, circular irradiation target area on a copy paper and then exposed to the following means of radiation at room temperature. The results of this experiment are reported in following Table I.

TABLE I

| Source of Energy | Dosage | Color Change from Yellow |
|---|---|---|
| UV light | — | dark blue* |
| 60 Cobalt γ-ray | 5 kGy | dark blue |
| X-ray | 1 kGy | blue |

*the transparent yellow dye did not absorb UV light; thus the polyacetylene monomer was immediately activated.

### EXAMPLE 2

A target area of Xerox copy paper was printed with yellow ink containing pentacosa-10,12-diynoic acid and then overprinted with a UV absorbing magenta dye to provide a red color. The samples were irradiated with the same energy sources as in example 1 and results are shown in Table 11. The fact that the image lost its sensitivity to UV is due to the absorbency at the particular wavelength contributed by the magenta dye. Such overprint affords increased light stability of the label. It was also noted that compared to the yellow images, the red targets resulted in much increased in the color change resulting from the radiation exposure.

TABLE II

| Source | Dose | Color (Color Change) |
|---|---|---|
| UV | — | Red (No Change) |
| X-Ray | 1 kGy | Reddish Black |
| 60Co Gamma Ray | 5 kGy | Black |

### EXAMPLE 3

The procedure described in Example 1 was repeated except that 5,7-dodecodiyne-1,12-bis(n-butyl carbamate), DDBC, was used to replace pentacosa-10,12-diynoic acid and 5 grams of DDBC was used with only 10 grams of the UV absorbent yellow dye. The results from UV, Co γ-ray and X-ray exposures are reported in the following Table III.

TABLE III

| Source | Dose | Color (Color Change) |
|---|---|---|
| UV | — | Very dark blue |
| X-Ray | 1 kGy | Blue |
| 60Co Gamma Ray | 5 kGy | Very dark blue |

### EXAMPLE 4

A target area of the paper was printed with yellow ink containing 5,7-dodecodiyne-1,12-bis(n-butyl carbamate) and overprinted with magenta dye to provide a red color. The samples were irradiated with the same energy sources as above in Example 3 and results are shown in following Table IV. Similar increase in light stability and image contrast was also observed.

TABLE IV

| Source | Dose | Color (Color Change) |
|---|---|---|
| UV | — | Red (No Change) |
| X-Ray | 1 kGy | Black |
| 60 Co Gamma Ray | 5 kGy | Black |

### EXAMPLE 5

10 grams of polyethylene glycol (Mw. 14,000) was melted and temperature was raised and maintained at 125° C. 1 gram of 5,7-dodecodiyne-1,12-bis(n-butyl carbamate) was added and mixed to provide a homogeneous solution. The solution was allowed to cool and solidify. The resulting solid was then mixed with 20 grams of water to dissolve polyethylene glycol and to form a stable dispersion. The radiochromic sensitive ink dispersion was then poured into an empty Epson black ink cartridge and the. Target areas were printed with the radiochromic ink using Epson Stylus 600. Color darkening was observed after exposure of the printed areas with radiation of the energy sources mentioned in the previous examples.

### EXAMPLE 6

A yellow target area described in Example 3 was compared to color bands of a separate vertical column illustrating gradations of darkening color from yellow to blue to black. Upon radiation with x-ray, the original yellow color turned blue at 1 kGy before turning black at dose higher than 5 kGy. This comparison provides means not only to ensure a sufficient radiation to effect disinfection (1 kGy) but also to prevent possible damage of a package contents resulting from radiation overexposure.

It will be understood that many alterations and substitutions can be made in the above examples without departing from the scope of this invention. For example many other printer ink dyes, such as magenta, red or green dyes, or their mixtures, can be substituted in the above examples to provide the contrasting color phase changes. Also, the dosage, energy and duration of exposure, as well as the active polyacetylene, can be altered or substituted to meet the limitations or requirements of certain packages or group of packages. Also the methods and types of printing can be changed to meet the requirements of the specific application situation.

What is claimed is:

1. A package or container having an irradiation target area containing a radiation sensitive indicia which comprises a normally crystalline, conjugated, radiochromic polyacetylene monomer containing 12 to 60 carbon atoms, which polyacetylene is capable of undergoing a visually distinguishable color alteration upon exposure to radiation at between about 1 KeV and about 100 MeV at a dosage of from 0.001 to about 100,000 Gy.

2. The package or container of claim 1 wherein said radiochromic polyacetylene monomer has the formula
A-(CH₂)ₘ—(C≡C—)ₚ—(CH₂)ₙ—B
wherein m and n each independently have a value of from 0 to 30; p has a value of from 2 to 4; A and B independently are each R, OR₁, OH, COOR₂, CONR₃R₄ or (CH₂)ᵣ—O—CO—NR₅R₆ and metal salts of the foregoing, where r has a value of from 1 to 4 and each of R, R₁, R₂, R₃, R₄, R₅ and R₆ are independently hydrogen or C₁ to C₁₂ alkyl, aryl and a mixture of said polyacetylene monomers.

3. The package of claim 1 imprinted with a polyacetylene monomer is selected from the group consisting of pentacosa-10,12-diynoic acid and 5,7-dodecodiyne-1,12-bis(butyl carbamate).

4. The package of claim 1 imprinted with said polyacetylene in a composition comprising the polyacetylene monomer uniformly dispersed in an inert carrier selected from the group consisting of water, alcohol, glycol, natural or synthetic wax or mixture of waxes, paraffin and a synthetic resin.

5. The package of claim 4 imprinted with a polyacetylene monomer composition comprising said polyacetylene monomer dispersed in an inert liquid carrier wherein the concentration of the monomer is between about 1 and about 90 wt. %.

6. The package of claim 4 imprinted in the target area with said liquid polyacetylene monomer/carrier composition which irradiation target area additionally contains a dye in a concentration of between about 1 and about 10 wt. % with respect to the polyacetylene monomer concentration.

7. The package of claim 6 wherein the dye is a UV absorbent dye or dye mixture.

8. The package of claim 1 imprinted with an alternative message whereupon radiation of the sensitive area with energy from X-Rays, Gamma-Rays, electrons, neutrons and/or photons causes darkening sufficient to obliterate a portion of the message.

9. A method of determining the degree of radiation received by a package, which comprises imprinting said package with the indicia of claim 1 and intercalating the dosage of irradiation with the degree of color change in the polyacetylene monomer upon exposure to irradiation.

10. The method of detoxifying a package which comprises imprinting said package with the indicia of claim 1 and exposing said imprinted package to irradiation sufficient to inactivate a harmful pathogen.

11. The method of detoxifying a package of claim 10 wherein said package is an item of mail.

12. The method of detoxifying the contents of a package of claim 10.

13. The method of claim 12 wherein said contents is a contaminated food, blood or blood component.

14. The method of altering the genetic or biological function of a living species contained in a package by exposing the package of claim 1 to radiation at between about 1 KeV and about 100 MeV at a dosage of from about 0.001 to 100,000 Gy.

15. The method of detoxifying a package which comprises imprinting the package with the indicia of claim 2 and exposing said imprinted package to irradiation sufficient to inactivate a harmful pathogen.

16. The method of detoxifying a package which comprises imprinting the package with the indicia of claim 5 and exposing said imprinted package to irradiation sufficient to inactivate a harmful pathogen.

17. The method of detoxifying a package which comprises imprinting the package with the indicia of claim 6 and exposing said imprinted package to irradiation sufficient to inactivate a harmful pathogen.

18. The method of detoxifying a package which comprises imprinting the package with the indicia of claim 7 and exposing said imprinted package to irradiation sufficient to inactivate a harmful pathogen.

19. The method of claim 10 wherein said package is irradiated at from about 1 MeV to about 10 Mev at a dosage of between about 1 to 50,000 Gy.

20. The process of applying the radiation sensitive indicia of claim 1 to a package which comprises imprinting the package by the use of a printing device, an ink stick, marker, crayon or a spray gun.

21. The process of claim 20 wherein the liquid radiation sensitive composition containing said polyacetylene monomer is applied by the use of a printer.

22. The process of claim 20 wherein the radiation sensitive indicia is applied to a stamp or label subsequently affixed to the package.

23. The process of preparing the indicia of claim 2 in a liquid composition, which comprises mixing, said crystalline polyacetylene monomer with an inert liquid carrier to form a dispersion.

24. The process of preparing the indicia of claim 2 in a liquid composition which comprises dissolving said crystalline polyacetylene in a solvent.

25. The process of preparing the indicia of claim 2 in a liquid composition which comprises dissolving said crystalline polyacetylene in a solvent before mixing with an inert liquid carrier.
